(19) United States
(12) Patent Application Publication
(10) Pub. No.: US 2023/0193254 A1
(43) Pub. Date: Jun. 22, 2023

(54) TOTAL RNA PROFILING OF BIOLOGICAL SAMPLES AND SINGLE CELLS

(71) Applicants: Chan Zuckerberg Biohub, Inc., San Francisco, CA (US); The Board of Trustees of the Leland Stanford Junior University, Stanford, CA (US)

(72) Inventors: Alina Isakova, Stanford, CA (US); Norma Neff, San Francisco, CA (US); Stephen R. Quake, San Francisco, CA (US)

(73) Assignees: Chan Zuckerberg Biohub, Inc., San Francisco, CA (US); The Board of Trustees of the Leland Stanford Junior University, Stanford, CA (US)

(21) Appl. No.: 17/999,158

(22) PCT Filed: May 20, 2021
(86) PCT No.: PCT/US21/33465

§ 371 Date: Nov. 17, 2022

Related U.S. Application Data
(60) Provisional application No. 63/027,825, filed on May 20, 2020.

(51) Int. Cl. C12N 15/10; C12P 19/34; C12Q 1/686; C12Q 1/6806

(52) U.S. Cl. CPC C12N 15/1096; C12P 19/34; C12Q 1/686; C12Q 1/6806

(57) ABSTRACT

Methods and materials for preparing DNA complementary to poly(A)-minus RNA are provided. The method includes conducting a template switching reaction by contacting a RNA-cDNA intermediate with a template switching oligonucleotide (TSO), extending the cDNA strand to include sequence complementary to the TSO and degrading the TSO. The method is capable of assaying a broad spectrum of coding and non-coding RNA from a single cell.

### CROSS-REFERENCE TO RELATED APPLICATIONS

This application claims priority to U.S. provisional application No. 63/027,825, filed May 20, 2020, the entire content of which is incorporated herein by reference.

### BACKGROUND

Efforts in characterizing transcriptional states of single cells have so far mostly focused on protein-coding RNA¹⁻⁴. However, a growing number of studies indicate that non-coding RNAs (ncRNAs), are actively involved in cell function and specialization⁵⁻⁸. Importantly, compared to the coding RNA, which is transcribed from only ˜2% of the genome, the non-coding RNA constitute a major fraction of all cellular transcripts and covers ˜70% of the genomic content⁹. The role of these transcripts in shaping different cell types and states remain poorly understood.

Several groups have demonstrated the possibility of measuring the levels of ncRNA in single cells^{10,11}. The respective methods, however, are designed to target only a subset of non-coding transcripts, which are either short (˜18-200 nt, e.g. microRNA)^{11,12} or long (>200 nt, e.g. IncRNA or circRNA)^{10,13-15}, while none of them offer a simultaneous assessment of all RNA types within a cell. This limits one's ability to map the regulatory connection between coding, and different types of non-coding transcripts within a cell and calls for the development of novel single-cell technologies capable of assaying both poly(A)⁺ and poly(A)⁻ RNA, irrespective of transcript length.

Template switching reactions are used to produce cDNA libraries or for characterization of mRNA populations. In one conventional approach, oligo(dT) fused to a first defined sequence serves as a primer to initiate reverse transcription from the poly(A) tail of an mRNA template, resulting in a first cDNA strand complementary to the RNA template. The reverse transcriptase enzyme (often MMLV) is able to “switch” templates from the 5′ end of the mRNA template to the 3′ end of a template-switching oligo (TSO). The TSO includes a known sequence, the complement of which is incorporated into the cDNA strand, sometimes called the “extended” first cDNA strand. This results in a cDNA flanked by predefined sequences, which may include binding sites for amplification primers, for example. The cDNA can be used as a template for amplification, library construction, and massively parallel sequencing. See, e.g., Zhu et al., 2001, “Reverse transcriptase template switching: a SMART approach for full-length cDNA library construction” BioTechniques 30:892-897; Wulf et al., 2019, “The template switching bias is marginally affected by the internal RNA sequence” J. Biol. Chem. 294:18220-18231.

### BRIEF DESCRIPTION OF THE INVENTION

In one aspect, disclosed is a method for preparing DNA complementary to poly(A)-minus RNA by i) treating the poly(A)-minus RNA with poly(A) polymerase (PAP) to add a 3′ poly(A) tail to the poly(A)-minus RNA, thereby producing poly(A)-plus RNA; ii) annealing a cDNA synthesis primer comprising oligo(dT) to the poly(A)-plus RNA produced in (i), and synthesizing a first cDNA strand, thereby producing an RNA-cDNA intermediate; iii) conducting a template switching reaction by contacting the RNA-cDNA intermediate with a template switching oligonucleotide (TSO) under conditions suitable for extension of the first cDNA strand, rendering the first cDNA strand additionally complementary to the TSO, wherein the TSO comprises at least two deoxyuridine nucleotides; and iv) degrading the TSO. In some approaches the TSO is degraded by treatment with uracil DNA glycosylase (UDG), and optionally with a combination of UDG and endonuclease VIII.

In various approaches the method further includes (v) amplifying sequence from the first cDNA strand to produce a pool of amplicons. In one approach the method further includes (vi) depleting high-abundance RNA species from the pool of amplicons. The high-abundance RNA species may include ribosomal RNA.

In some approaches in step (i) a mixture comprising poly(A)-minus RNA and polyadenylated RNA is treated with poly(A) polymerase (PAP) to add a 3′ poly(A) tail to the poly(A)-minus RNA and to the polyadenylated RNA, thereby producing the poly(A)-plus RNA. The mixture may include RNA from one single cell, optionally a human cell. The mixture may be total RNA from a single cell. The mixture comprises RNA from human cells.

In some approaches the step of amplifying comprises associating the sequence from the first cDNA strand with one or more sequence elements selected from adaptors, indexing sequences, oligonucleotide binding sequences and barcodes. The method may include sequencing amplicons produced in step (v). In some approaches, steps (i)-(iv) or steps (i)-(v) are carried out in the same compartment, and optionally cell lysis prior to step (i) is carried out in the same compartment.

In one aspect disclosed is a method for preparing DNA complementary to poly(A)-minus RNA comprising the steps of i) treating the poly(A)-minus RNA with polynucleotide transferase to add a homopolynucleotide poly(N) at the 3′ends, where poly(N) is selected from poly(A), poly(C), poly(G) and poly(U), thereby producing poly(N)-plus RNA; ii) annealing a cDNA synthesis primer comprising oligo(dN′) to the poly(N)-plus RNA produced in (i), wherein N′ [“N prime”] is a nucleotide that basepairs with N, and synthesizing a first cDNA strand, thereby producing an RNA-cDNA intermediate; iii) conducting a template switching reaction by contacting the RNA-cDNA intermediate with a template switching oligonucleotide (TSO) under conditions suitable for extension of the first cDNA strand, rendering the first cDNA strand additionally complementary to the TSO, wherein the TSO comprises at least two (2) deoxyuridine nucleotides; and iv) degrading the TSO. The template switching oligonucleotide (TSO) may have at least two (2) deoxyuridine nucleotides and, optionally, at least two ribonucleotide residues, e.g., adenosine residues. In some cases the TSO is 30 to 50 nucleotides in length and includes three (3) to ten (10) deoxyuridine nucleotides. In some cases the TSO includes rGrG+G at its 3′ end, where +G is a locked nucleic acid. In some cases, the TSO is biotinylated at the 5′ terminus. In some approaches a TSO described herein has at least 2, at least 3, at least 4, at least 5, at least 6, at least 7, at least 8, at least 9 , or at least 10 deoxyuridine nucleotides; and/or at least 10%, at least 15% or at least 20% of the nucleotides in the TSO are deoxyuridine; and/or iii) deoxyuridine nucleotides are spaced or positioned in the TSO such that following fragmentation of the TSO by deamination of all uridine nucleotides no remaining fragment of the TSO is longer than 10 nucleotides, no remaining fragment of the TSO is longer than 9 nucleotides, or no remaining fragment of the TSO is longer than 8 nucleotides.

Also disclosed is a method of identifying or distinguishing human cell types based on the abundance of non-coding transcripts by (i) determining an expression profile of at least one, preferably at least two, poly(A)-minus RNA type selected from miscRNA, IncRNA, snoRNA, miRNA, snRNA and tRNA in a cell(s) and (ii) matching the profile to a reference profile characteristic of a human cell type.

### BRIEF DESCRIPTION OF THE FIGURES

FIG. 1. a. and c. Schematic comparison of Smart-seq2 and Smart-seq-total pipelines. Following cell lysis, total cellular RNA is polyadenylated, primed with anchored oligodT and reverse transcribed in a presence of the custom degradable TSO. After reverse transcription TSO is enzymatically cleaved, single-stranded cDNA is amplified and cleaned up. It is then indexed, pooled and depleted from ribosomal sequences using DASH³³. b. Mean number of genes per biotype detected by Smart-seq2 and Smart-seq-total in single HEK293T cells. Results for smart-seq-total are presented as the upper bar in each biotype and results for smart-seq2 are presented as the lower bar. Genes were assigned to a specific biotype based on GENCODE v32 annotation for the reference chromosomes. tRNA was quantified using high-confidence gene set obtained from GtRNAdb. Libraries were depth normalized to ˜2.5 Mio reads per cell. Error bars denote standard deviation (n=4).

FIG. 2. t-SNE (t-distributed stochastic neighbor embedding) plots of three profiled human cell types generated using indicated subset of genes. Fibroblasts (F), HEK293T (H) and MCF7 (M). From left to right: protein coding, IncRNA, miRNA and other small ncRNA (include snoRNA, snRNA, scaRNA, scRNA and miscRNA). We have excluded histone coding genes from protein coding (polyA+) set, since a large fraction of these RNAs are known to lack polyA tail³⁴.

FIG. 3. a. Exemplary coding and non-coding genes that are up- or downregulated during EB formation. b. UMAP plot of collected cells shaded by timepoint. Cell were clustered using k-nearest neighbor algorithm and cell lineages were annotated based on marker genes expressed within identified clusters.

FIG. 4 a. Sequencing scheme of a scRNA-seq library prepared with Smart-seq-total b. Bioanalyzed traces showing the fragment sizes of amplified cDNA prepared from single cells using Smart-seq-total with and without TSO removal step. c. Mean number of genes per biotype detected by Smart-seq2 and Smart-seq-total in single HEK293T cells. Same as FIG. 1b but for tRNA, miscRNA, TEC, intronic and antisense transcripts. Results for smart-seq-total are presented as the upper bar in each biotype and results for smart-seq2 are presented as the lower bar.

FIG. 5. a. Number of counts and number of genes per cell grouped by RNA type. Computed based on 612 profiled cells. b. Number of genes, number of counts as well as the percentage of mitochondrial and histone RNA per cell computed for fibroblasts, HEK293T and MCF7 cells. c. Number of detected genes by RNA type in three profiled cell types. For each RNA type, data are presented in the order, from left to right, Fibroblasts-HEK293T-MCF7.

FIG. 6. Dot plots of marker genes identified in fibroblasts, HEK293T and MCF7 cells grouped by RNA type.

### DETAILED DESCRIPTION

The ability to interrogate total RNA content of single cells would enable better mapping of the transcriptional logic behind emerging cell types and states. However, current RNA-seq methods are unable to simultaneously monitor both short and long, poly(A)+ and poly(A)− transcripts at the single-cell level, and thus deliver only a partial snapshot of the cellular RNAome. Here, we describe “Smart-seq-total” (also referred to as “Smart-seq4”), a method capable of assaying a broad spectrum of coding and non-coding RNA from a single cell. Built upon the template-switch mechanism, Smart-seq-total bears the key feature of its predecessor, Smart-seq2¹⁶, namely, the ability to capture full-length transcripts with high yield and quality. It also outperforms current poly(A)-independent total RNA-seq protocols by capturing transcripts of a broad size range, thus, allowing us to simultaneously analyze protein-coding, long non-coding, microRNA and other non-coding RNA from single cells.

We used Smart-seq-total to analyze the total RNAome of human primary fibroblasts, HEK293T and MCF7 cells as well as that of induced murine embryonic stem cells differentiated into embryoid bodies. We show that simultaneous measurement of non-coding RNA and mRNA from the same cell enables elucidation of new roles of non-coding RNA throughout essential processes such as cell cycle or lineage commitment. Moreover, we show that cell types can be distinguished based on the abundance of non-coding transcripts only.

Smart-seq-total is a scalable method, designed to capture both coding and non-coding transcripts irrespective of their length. This method harnesses template switching capability of MMLV (Moloney murine leukemia virus) reverse transcriptase to generate full-length cDNA with high yield and quality. In addition, Smart-seq-total is designed to capture non-polyadenylated RNA through template-independent addition of polyA tails and further oligo-dT priming of all cellular transcripts. Therefore, Smart-seq-total simultaneously measures cellular levels of mRNA alongside other RNA types in the same cell, which permits the discovery of non-coding regulatory patterns of a cell and at the same time facilitates the integration of this data with the existent single cell RNA-seq datasets.

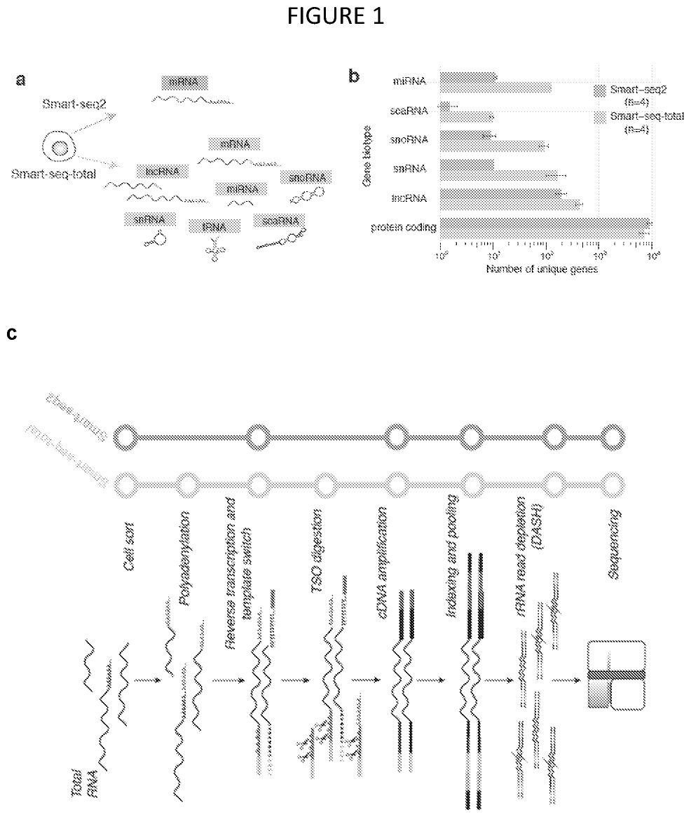

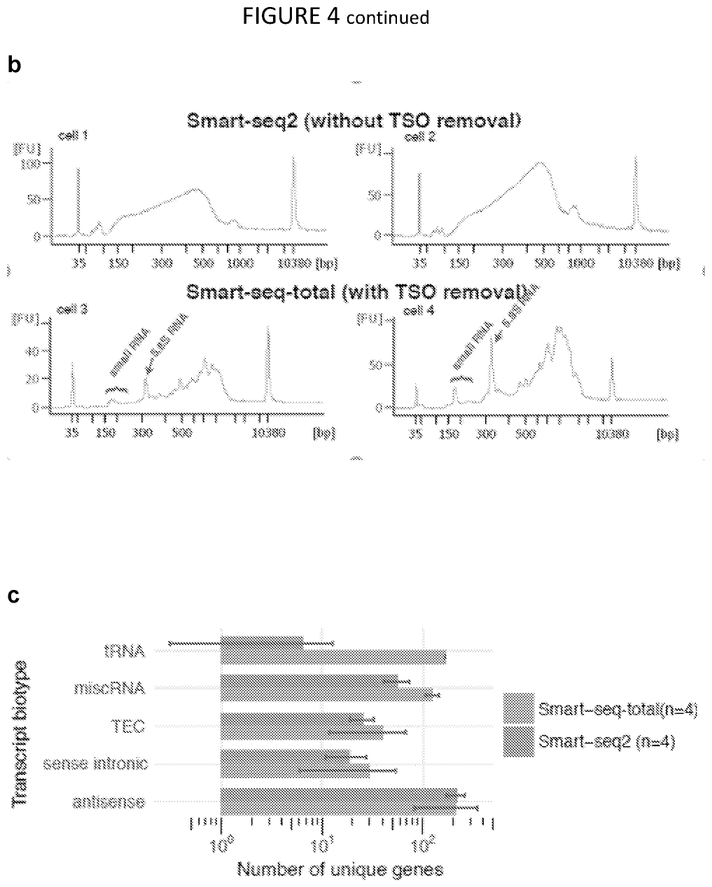

Smart-seq-total relies on the ability of E.coli poly(A) polymerase to add adenine tails to the 3′ prime of RNA molecules. Total polyadenylated RNA is then reverse transcribed using anchored oligo dT, in the presence of the template switch oligo (TSO)¹⁷ (FIG. 1a). Compared to previous studies that explored similar approaches to construct libraries from total RNAs^{18,19}, Smart-seq-total utilizes an optimized version of the TSO¹⁶, specifically engineered to be rapidly eliminated from the reaction directly following the reverse transcription. This allows us to remove the “contaminant” constructs, originating from polyA-tailing and mispriming of TSO which otherwise dominate the resulting sequencing library and render the short RNA undetectable (see FIG. 4a-b). Additionally, we employ a CRISPR-mediated removal of overrepresented sequences, which allows us to eliminate the majority of the sequences corresponding to ribosomal RNA from the final library in a single-pool reaction (targeting 69 regions).

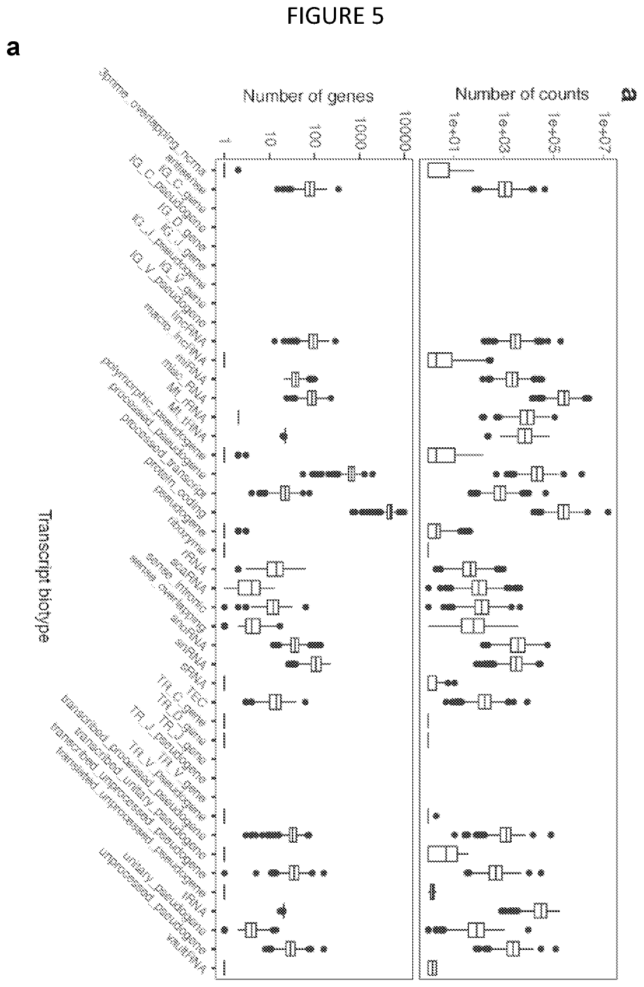

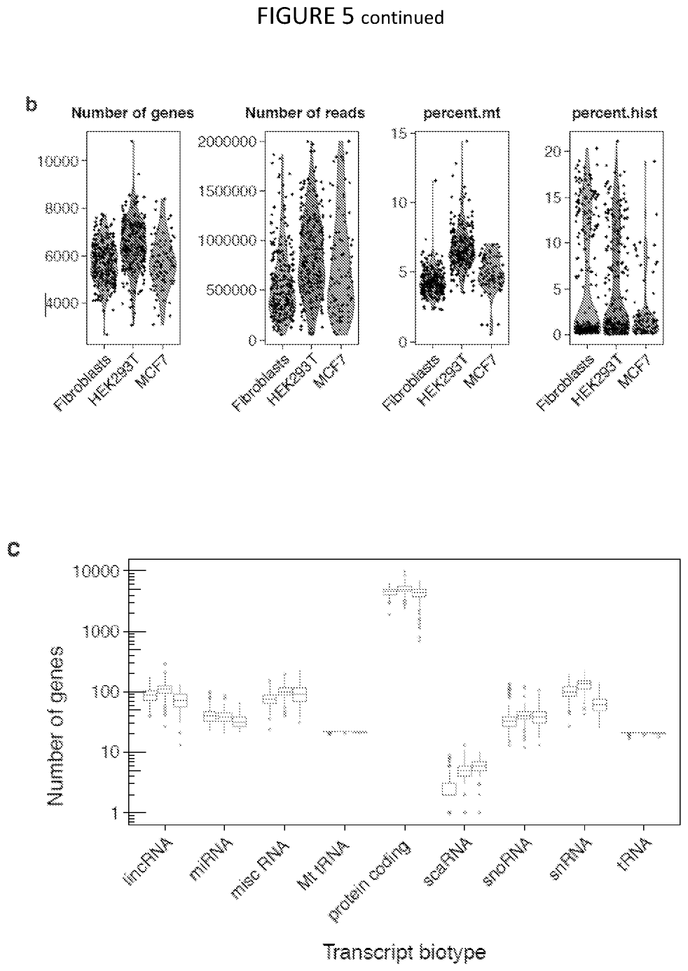

Applied to single HEK293T cells Smart-seq-total identified, alongside mRNA, histone RNA and a broad spectrum of non-coding RNA genes, such as snoRNA, scaRNA and IncRNA. A majority of these molecules endogenously lack poly(A) tails and thus cannot be captured through a direct polyA-priming employed by Smart-seq2 or other popular scRNA-seq methods¹ (FIG. 1b). Among other ncRNA, detected uniquely by Smart-seq-total are tRNAs and mature miRNAs (FIG. 1b; FIG. 5).

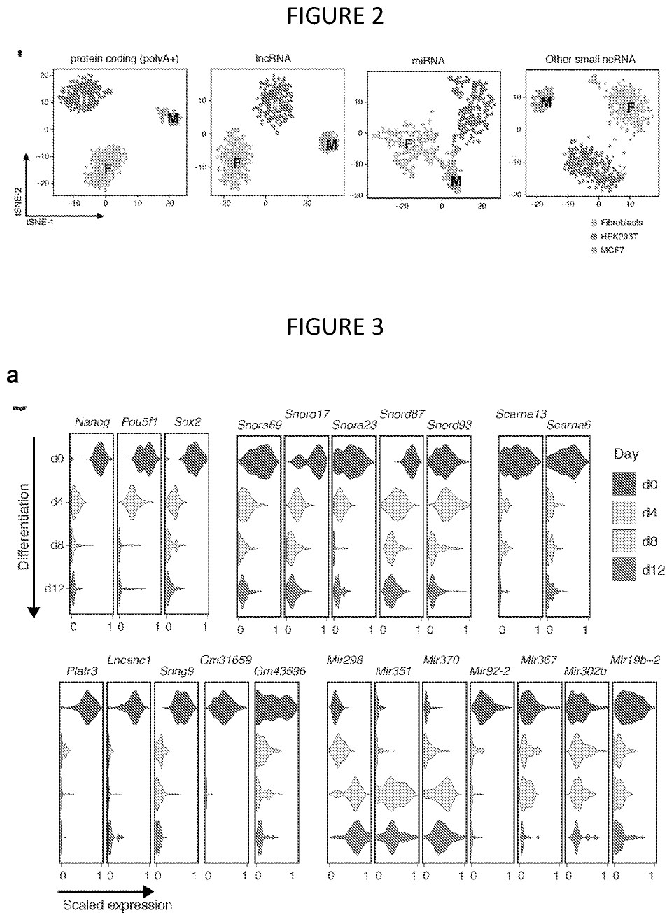

To assess the scalability of the method, we sequenced total RNA from individual human primary dermal fibroblasts (n=277), HEK293T (n=245) and MCF7 (n=90) cells sorted in 384-well plates and processed in 1/10 of the standard Smart-seq2 volume¹⁶. Within all three cell types we identified a broad spectrum of transcripts such as mRNA, miRNA, IncRNA, snoRNA in each profiled cell (FIG. 2). We found metazoan cytoplasmic RNA7SK and RN7SL1, annotated as ‘miscellaneous RNA’ type (miscRNA) in GENCODE database, to be the most abundant in our data comprising together ˜40% of all mapped reads (see Table 1). Numbers represent percentage of total.

TABLE 1

| | Fibroblasts | HEK293T | MCF7 |
|---|---|---|---|
| protein coding | 51 | 50 | 43 |
| miscRNA | 42 | 44 | 55 |
| IncRNA | 1 | 1 | 1 |
| snoRNA | 1 | 0.5 | <0.5 |
| miRNA | 0.4 | 0.5 | <0.5 |
| snRNA | 1 | 1 | <0.5 |
| tRNA | 4 | 4 | 1 |

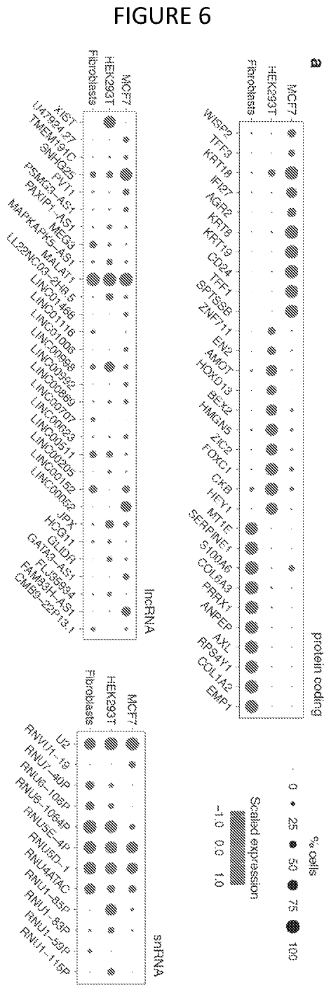

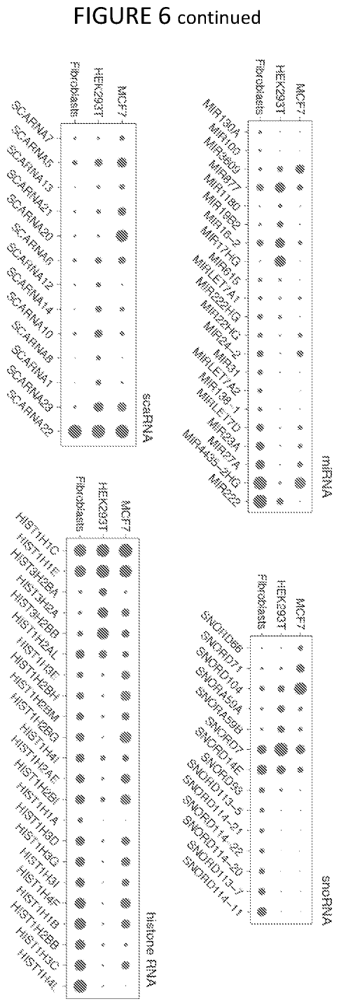

Among cell-type specific transcripts we found well-characterized marker genes for either fibroblasts (COL1A2, FN1, MEGA HEK293T (CKB, AMOT, HEY1) or MCF7 cells (KRTB, TFF1) as well as transcripts which belong to various types of ncRNA, such as microRNA, snoRNA and IncRNA. See FIG. 6. For example, we found high levels of M1R222 in fibroblasts while could not detect it in MCF7 cells. We also observed that oncogenic miRNA cluster MIR17HG is specific to HEK293T cells, while not found in neither fibroblasts nor MCF7 cells. In contrast, MCF7-specific transcripts include IncRNA, such as LINC00052, as well as snoRNA, such as SNORD 71 and SNORD104.

Given observed differences in the levels of non-coding RNA across profiled cells, we next asked whether non-coding RNA alone could be used to distinguish cell types. To answer this question, we performed principal component analysis (PCA) followed by the dimensionality reduction on the genes corresponding to one or multiple ncRNA types. Evaluation of the similarity between cells in two-dimensional space reveled that, taken alone, IncRNA, and miRNA separate the investigated cell types in three distinct clusters, while combining snoRNA, scaRNA, snRNA and tRNA together allowed us to achieve similar results (FIG. 2).

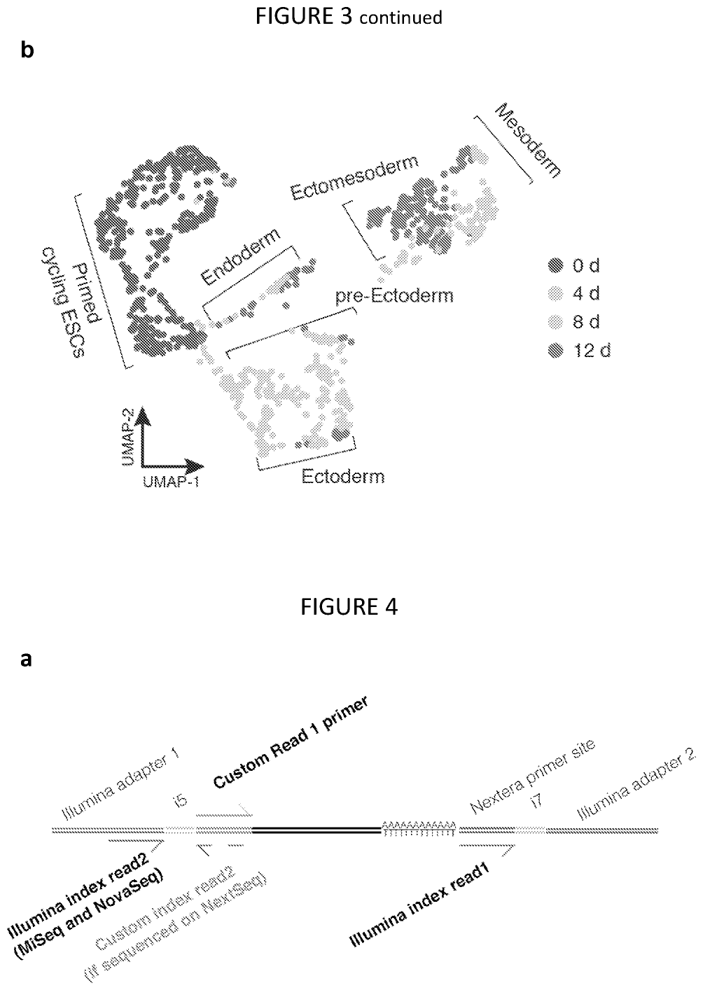

In addition to cell-type dependent differences in ncRNA, the abundance of certain non-coding transcripts also changed throughout the cell cycle (FIG. 3a). In agreement with previous bulk studies, suggesting the involvement or miRNA in cell-cycle regulation^{20,21}, we found that levels of a subset of miRNAs in a cell dynamically change through the cell cycle, peaking at either S, G2M or G1 phase (FIG. 3a). For example, our data showed that the levels of MIR16-2 in fibroblasts are high during S phase and later gradually decrease during G2M and G1 phases (data not shown). The opposite holds true for M1R222 in HEK293T cells, which is upregulated during cell proliferation (G1) and gradually decays during DNA replication (S) and cell division (G2M) phases (data not shown). Among miRNAs upregulated during G2M phase we identified MIR27A, MIR103A2, MIRLET7a and M1R877 (data not shown). In addition to miRNA, a large number of IncRNA, snRNA, scaRNA snoRNA and miscRNA were also upregulated during the G2M phase. Given the active role of these RNA types in splicing and ribosome biogenesis, we suggest they are produced by a cell in response to a rapid demand for protein synthesis and cell growth during G2M phase.

To further link the observed non-coding RNA dynamics with the expression of well-characterized cell-cycle mRNA markers, we searched for co-regulated coding and non-coding genes throughout the cell cycle. We identified 24 clusters comprised of co-expressed coding and non-coding genes specific to either one or multiple cell types. Two of these mixed-gene clusters (27 genes upregulated in S phase and 47 genes upregulated in G2M phase) showed identical patterns in all three profiled cell types. Interestingly, both clusters are marked by landmark cell-cycle genes, such as CDK1, MCM2 and TOP2A, but also include miRNAs, IncRNAs and snRNAs previously unknown to follow a distinct expression pattern upon transition between phases.

Histone RNA is another type of mainly non-polyadenylated RNA which we observed to be strongly correlated with the cell cycle. Consistent with prior studies^{22,23}, histone RNA levels sharply rise during S-phase in all three profiled cell types. The ability to capture non-polyadenylated histones also has a strong impact on cell clustering, by introducing a cell cycle bias. Particularly, histones drive the separation of each cell type in two distinct populations, marked by increased levels of certain histone genes in response to DNA replication.

In addition to being expressed in a cell cycle-dependent manner, we also identified several histones to be cell type specific. For example, HIST1H4L is expressed in fibroblasts but absent in HEK293T and MCF7 cells, while HIST1H1B, is absent in HEK293T cells while present in two other cell types. Given the importance of histones in establishing and maintaining a distinct chromatin landscape of a cell, we anticipate that the ability to measure corresponding transcripts could be valuable for predicting epigenetic state of a cell.

Finally, we sought to understand whether the unique non-coding signature acquired by different cell types is established during early stages of cell development and if so, how dynamic it is with respect to cellular transcriptome. To address this question we referred to an in vitro model of early lineage commitment: the differentiation of pluripotent stem cells into embryoid bodies²⁴. The role of ncRNA in maintaining stem cell pluripotency and lineage commitment has been demonstrated previously through bulk experiments^{25.26}. Thus, we hypothesized that applying Smart-seq-total to single cells at different stages of embryoid body (EB) formation would allow us to identify co-expressed coding and non-coding transcripts within emerging lineages. As such, we analyzed the RNAome of primed pluripotent stem cells (d0) and that of individual cells obtained from dissociated embryoid bodies at days 4 (d4), 8 (d8) and 12 (d12) of culture. Table 2 shows distribution of mapped reads across RNA biotypes. Genes were assigned to a specific biotype based on GENCODE M23 annotation for the reference chromosomes. tRNA was quantified by mapping the reads, non-mapping to any other RNA type, to high-confidence gene set obtained from GtRNAdb. Numbers represent percentage of total.

TABLE 2

| RNA Biotype | d 0 | d 4 | d 8 | d 12 |
|---|---|---|---|---|
| protein coding | 65 | 50 | 53 | 54 |
| miscRNA | 25 | 32 | 34 | 34 |
| IncRNA | 5 | 7 | 3 | 3 |
| snoRNA | 1 | 1 | 1 | 1 |
| miRNA | 2 | 6 | 6 | 7 |
| snRNA | 1 | 2 | 1 | 1 |
| tRNA | 1 | 3 | 2 | 2 |

We found that the fraction of mRNA with respect to all other analyzed transcripts was higher in pluripotent compared to differentiated cells (65% vs 50-54%). Consistent with previous studies²⁷, the number of coding genes expressed by pluripotent stem cells was also higher compared to differentiated progenitors. This was also the case for several non-coding RNA types, such as IncRNA, miRNA and scaRNA. Specifically, we observed that the levels of certain snoRNAs (such as Snord17, Snora23, Snord87), scaRNAs (such as Scarna13 and Scarna6), IncRNAs (Platr3, Lncenc1, Snhg9, Gm31659, etc.) and miRNAs (Mir92-2, Mir302b, Mir19b-2) go down after cells exit pluripotency (FIG. 4b). In contrast, we also identified that the levels of several IncRNAs (Tug1, Meg3, Lockd) and miRNAs (Mir298, Mir351, Mir370) increase with differentiation (FIG. 3a).

Louvain clustering of all collected cells, revealed the presence of six molecularly distinct populations which we assigned to: primed mESC, pre-Ectoderm, Ectoderm, Endoderm, Ectomesoderm and Mesoderm (FIG. 3b), based on the expression of know lineage-specific marker genes (e.g. Nanog and Pou5f1 for pluripotent cells, Pax6 and Olfr787 for ectoderm, Afp and Shh for endoderm, Acta2 and Col3a1 for mesoderm²⁸) (data not shown). The analysis of genes differentially expressed between primed mESCs and each of the identified clusters showed that in addition to well-characterized lineage-specific mRNAs (data not shown)^{29,30} and IncRNAs (Tug1 in ectodermal and Meg3 mesodermal lineages respectively)⁶, other ncRNA genes such as miRNAs, scaRNAs, snoRNAs, tRNAs and histone RNAs are either specifically expressed or downregulated within a certain lineage.

We next used PAGA³¹ to infer a developmental trajectory and compute pseudotime coordinates for each cell in our data (see Methods). Aligning cells in pseudotime within each lineage further confirmed the existence of expression gradient within different RNA types (FIG. 44. Furthermore, we found that the majority of identified variable non-coding transcripts were germ-layer specific. Examples of such transcripts include Mir2137, Mir320, Gm49024 and Gm38708 in ectoderm, Mir351, Mir370 and Meg3 in mesoderm as well Neat1 in endoderm. Mir296 and Mir298 were expressed in both mesoderm and endoderm but absent in ectoderm.

Finally, to understand the relationship between mRNA and non-coding RNA genes we performed a pairwise correlation analysis of gene expression across all sampled cells. We found that the expression of ˜50% of identified histone-coding genes correlated with the expression of other protein-coding genes (Spearman rho >0.5). In addition, we found that multiple ncRNAs from all assayed RNA types (e.g. miRNA, snoRNA, snRNA, etc.) are positively correlated with the expression of protein-coding genes. Most of these ncRNAs represent putative uncharacterized regulators of linage commitment that require further validation through loss-of-function experiments.

Altogether, Smart-seq-total enables an unbiased exploration of a broad spectrum of coding and non-coding RNA in individual cells. We anticipate Smart-seq-total to facilitate the identification of non-coding regulatory patterns and its functional role in regulating cellular functions and shaping cellular identity. This also means shifting the current protein-centered view on gene regulation towards comprehensive maps featuring both, protein and RNA regulators.

In one aspect, the invention provides a method of identifying one or more human cell types or tissue types in sample, or distinguishing human cell types of tissue types from each other, based on the abundance of non-coding transcripts. In one approach the method includes determining an expression profile of at least one, preferably at least two, poly(A)-minus RNA types from the cells. In one approach the expression profile may be compared to expression profile(s) obtained from other cells or tissues obtained from the same person or individual, from a subject of the same species. In one approach the expression profile is compared to a reference profile or database of reference profiles characteristic of specific cell or tissue types. In one approach the poly(A)-minus RNA types are one or more of miscRNA, IncRNA, snoRNA, miRNA, snRNA and tRNA in a cell(s), or alternatively one or more of IncRNA, snoRNA, miRNA, and snRNA.

### Smart-seq2

As discussed above, Smart-seq-total shares key features with Smart-seq2. Smart-seq-total differs from Smart-seq2, in part, by the inclusion of a polyadenylation step, use of a modified TSO, and the incorporation of DASH to remove ribosomal RNA and, optionally, other undesired high-abundance species. These innovations and improvements allow Smart-seq-total to capture transcripts of a broad size range (e.g., full-length transcripts) with high yield and quality.

Persons of ordinary skill in the art are familiar with Smart-seq2, including well known variations of individual steps. It will be within the ability of persons of ordinary skill in the art, guided by this disclosure, to incorporate the elements discussed herein into any “Smart-seq2” protocol. See, e.g., U.S. Pat. No. 10,266,894B2, “Methods and compositions for cDNA synthesis and single-cell transcriptome profiling using template switching reaction” incorporated herein by reference; and Picelli et al., 2013, “Smart-seq2 for sensitive full-length transcriptome profiling in single cells,” Nat Methods 10:1096-1098; Picelli et al., 2014, “Full-length RNA-Seq from single cells using Smart-seq2” Nat. Protocols 9:171-181, each of which is incorporated herein by reference. Also see Picelli, “Single-cell RNA sequencing: The future of genome biology is now” (2017), RNA Biol; 14(5): 637-650); Saliba et al., “RNA-seq: advances and future challenges (2014). Nucleic Acids Res; 42:8845-60. Smart-seq2; and Plessy et al., 2010, “Linking promoters to functional transcripts in small samples with nanoCAGE and CAGEscan,” Nat Methods 7:528-534. doi:10.1038, each of which is incorporated herein by reference.

### Single Cell Isolation

Nucleic acid analysis according to the invention can be carried out using RNA from a single cell or RNA from multiple cells. Generally total cellular RNA is used, but analysis of RNA fractions is also possible.

Several methods can be used to isolate individual cells and detailed description of cell isolation methods are available, for example, in Picelli, “Single-cell RNA sequencing: The future of genome biology is now” (2017), RNA Biol; 14(5): 637-650), and in Saliba et al., “RNA-seq: advances and future challenges (2014). Nucleic Acids Res; 42:8845-60. Examples include micromanipulation, the use of optical tweezers, Laser Capture Microdissection (LCM), and Fluorescence Activated Cell Sorting (FACS). Cells can be isolated or sorted based on a variety of properties, such as size, morphology, optical properties, cell surface markers (antigens), the presence, absence, or level of one or more specific polypeptides expressed by the cell. Microfluidic devices, such as the C1™ Single-Cell Auto Prep System (Fluidigm) are also available and allow automated capture of single cells using special Integrated Fluidic Circuits (IFC). Other methods, such as can also be used for single-cell isolation.

### RNA Isolation

Cellular RNA collected using art known means. In many applications total cellular RNA is obtained. However, specific RNA fractions, or total RNA depleted of certain fractions, may be used.

RNA may be obtained from any biological source, including isolated tissue or cells (e.g., single cells or, alternatively, small groups of cells, such as five or fewer cells), blood, urine or other body fluids). RNA may be purified or partially purified. In one approach RNA is obtained from a single cell, single cell lysate, or single cell soluble fraction without substantial purification (e.g., without purification or without purification beyond removal of insoluble cell components).

RNA may be obtained from any source, including prokaryotes (e.g., bacteria), eukaryotes, plants, animals, mammals (e.g., humans), viruses, and combinations of sources (e.g., RNA from a sample containing human cells and bacterial cells from human gut microbiome).

The Smart-seq-total method is highly sensitive and can be carried out using very small quantities of RNA, such as less than 500 pg, less than 250 pg, less than 125 pg, or less than 50 pg. A typical mammalian cell contains 10-100 pg total RNA, depending on the species, cell type, developmental stage and physiological state. The majority of RNA molecules are tRNAs and rRNAs. mRNA accounts for about 1-5% of the total cellular RNA.

### 3′ Polyadenylation and Addition of Other Homopolymers

RNA obtained from cells includes mRNA and poly(A)-minus RNA types such as miRNA, IncRNA, snoRNA, Y RNA, snRNA, SRP RNA, RNA7SK, RN7SL1 and piRNA, as well as tRNA and rRNA. See Seal et al., 2020, “A guide to naming human non-coding RNA genes” EMBO J. 39: e103777. In the Smart-seq-total protocol, a nucleotide homopolymer (e.g., pA, pC, pT or pG) is generated at the 3′ end of the RNAs. The homopolymer serves as a binding site for a reverse transcription primer, optionally an anchored primer.

In one approach RNA, e.g., total cellular RNA is polyadenylated, primed with anchored oligo dT, and reverse transcribed in a presence of the custom degradable TSO. Polyadenylation may be accomplished by treating and RNA-containing sample with an enzyme such as poly(A) polymerase (PAP), which is also called polynucleotide adenylyltransferase (EC 2.7.7.19). PAP catalyzes the template-independent addition of adenosine residues (e.g., from ATP) to the 3′-end of all classes of RNA with a 3′OH terminus. Many PAP enzymes suitable for the method are known. E. coli PAP and yeast PAP are commercially available (Thermo Scientific). Other PAPs include S. pombe (Rissland et al., 2007, “Efficient RNA Polyuridylation by Noncanonical Poly(A) Polymerase” Mol. Cell. Bio. 27:10:3612-24), and mammalian PAPs.

In alternative embodiments, the homopolymer is poly(C), poly(T) or poly(G), which may be added using alternative enzymes and/or reagents. For example, Schizosaccharomyces pombe Cid1 Poly(U) Polymerase catalyzes the template independent addition of UMP from UTP to the 3′ end of RNA and will add other NTPs at slower rates. See Wickens, M. and Kwak, J. E., 2008, Science 319, 1344. In these cases, the reverse transcription primer will be entirely or partly complementary to the homopolymer sequence.

### Reverse Transcription and Template Switch

RNA (e.g., total RNA from a single cell) is reverse transcribed using a MMLV reverse transcriptase (RT) or similar RT with the template switching capability. For example, the MMLV reverse transcriptase derivative SuperScript II (SSII; Life/Invitrogen^{47,48}) may be used. Betaine, MgCl, and other agents may be included to increase cDNA yield. The template switching oligo (TSO) is included in the reverse transcription reaction and, after annealing of the three (3) terminal nucleotides of the TSO with the about 3 (e.g., 2-5) cytosine extension generated by, e.g., MMLV, the reverse transcriptase extends the cDNA using the TSO as template.

As noted above, the first strand reverse transcription primer anneals to the homopolynucleotide (e.g., poly(A)) added to the polyadenylate-minus RNA. The primer may include other sequence elements as well, typically including a primer binding site used for amplification of the resulting cDNA, and sequencing primer binding sequences, adaptor sequences, barcodes, indexing sequences, and/or unique molecular identifier sequences.

In some approaches, a five-prime cap (5′ cap) is enzymatically added prior to the reverse transcription reaction. The 5′ cap is reported to improve the ability of MMLV reverse transcriptase to template switch. See Wulf et al., 2019, “The template switching bias is marginally affected by the internal RNA sequence” J. Biol. Chem. 294:18220-18231. A 5′ cap can be added by art-known and commercially available methods (see Shuman, S. (1990). J. Biol. Chem. 265, 11960-11966; New England Biolabs Cat. #M20805).

### TSO Structure

The 3′ end of the TSO generally comprises three riboguanosines (rGrGrG), or, more often, comprises two riboguanosines and one locked nucleic acid (LNA)-modified guanosine (rGrG+G) to facilitate template switching. In some cases the TSO is an RNA/DNA chimera or has isomeric bases (Kapteyn et al., 2010, “Incorporation of non-natural nucleotides into template-switching oligonucleotides reduces background and improves cDNA synthesis from very small RNA samples,” BMC Genomics, 11:413).

The upstream (5′) portion of the TSO may includes deoxyuridine (dU) nucleotides, as discussed below, and may also include additional sequences including amplification primer binding sequences, amplification sequencing primer binding sequences, adaptor sequences, barcodes, indexing sequences, and/or unique molecular identifier sequences. The length of the TSO is generally 30-50 nucleotides, including the three nucleotides at the 3′ terminus, often 35-45 nucleotides, but the TSO may be longer or shorter. In some embodiments the TSO has at least 2, at least 3, at least 4, at least 5, at least 6, at least 7, at least 8, or at least 9 deoxyuridine nucleotides, or at least 10 deoxyuridine nucleotides. In some embodiments at least 10%, at least 15% or at least 20% of the nucleotide in the TSO are deoxyuridine. In some embodiments at least 10%, at least 15% or at least 20% of the nucleotide in the TSO are deoxyuridine. In some embodiments deoxyuridine nucleotides are spaced or positioned in the TSO such that following fragmentation of the TSO by removal of uracil (assuming cleavage at all sites) no remaining fragment of the TSO is longer than 10 nucleotides, no remaining fragment of the TSO is longer than 9 nucleotides, no remaining fragment of the TSO is longer than 8 nucleotides, or no remaining fragment of the TSO is longer than 7 nucleotides.

An exemplary TSO has the structure: 5′-UCGUCGGCAGCGUCAGUUGUAUCAACUCAG ACAUrGrG+G-3′ [SEQ ID NO:1]. In some embodiments the TSO is biotinylated at the 5′end. An exemplary biotinylated TSO has the structure: 5′-biotin-UCGUCGGCAGCGUCAGUUGUAUCAACUCAGACAUrGrG+G-3′ [SEQ ID NO:1].

### TSO Degradation

Any method for degrading the TSO may be used.

UDG Treatment

Following the reverse transcription step, treatment with uracil DNA glycosylase (UDG), also known as uracil-N-deglycosylase (UNG), or with UDG in combination with endonuclease VIII (USER° Enzyme, New England Biolabs) removes uracil, resulting in rapid degradation of the TSO.

5′ Phosphorylated TSO and Degradation Using Exonuclease

In an alternative approach, the TSO comprises 5′-phosphate and a 5′ to 3′ exonuclease (e.g., lambda exonuclease), that specifically digests 5′phosphorylated oligonucleotides, is added to degrade the TSO.

### Amplification and Library Preparation

Following TSO digestion, the resulting cDNA may be amplified. In one approach, PCR primers bind the TSO sequence (or its complement) and the reverse transcription primer sequence (or its complement). In this step, the PCR amplification primer(s) can add primer binding sequences, adaptor sequences, restriction sites, barcodes, indexing sequences, and/or unique molecular identifier sequences. In some implementations amplification comprises a pre-amplification step, followed by an amplification step that added additional sequencing elements (e.g., indexing sequences). Optionally, following TSO digestion cDNA from multiple cells can be pooled prior to amplification. Optionally, following amplification, amplicons from multiple compartments can be pooled.

### Single Tube

Using the Smart-seq-total method, it is possible to carry out all steps from cell lysis to cDNA amplification in the same compartment (e.g., reaction tube, well, chamber, droplet, etc.) with no intermediate purification, transfer or fluid exchange required. In one approach, polyadenylation, reverse transcription and template switch, and TSO digestion are carried out in the same compartment. In one approach, polyadenylation, reverse transcription and template switch, TSO digestion and cDNA amplification. In some versions cell lysis prior to polyadenylation is also carried out in the same compartment.

FIG. 6b illustrates the effect of omitting rapid TSO degradation following polyadenylation, reverse transcription and amplification of total RNA in a single tube. As shown in the Figure, absent the degradation step, most or all of resulting product is contaminant products originated from the polyadenylation and priming from the TSO.

### Depletion of High Abundance Sequences

DASH (“Depletion of Abundant Sequences by Hybridization”) is used in the present method for depletion of high-abundance species, such as rRNA, when preparing sequencing libraries. See, e.g., Gu et al., 2016, “Depletion of Abundant Sequences by Hybridization (DASH): using Cas9 to remove unwanted high-abundance species in sequencing libraries and molecular counting applications,” Genome Biology 17:41 (httpslidoi.org/10.1186/s13059-016-0904-5). The DASH protocol may be designed to deplete unwanted high-abundance species other than, or in addition to, ribosomal RNA. For example, the DASH protocol may be used to deplete RNA transcripts of histone-encoding genes.

### Sequencing

The Smart-seq-total method can be adapted to any massively parallel sequencing (MPS) sequencing platform including methods described in Goodwin et al., 2016, “Coming of age: ten years of next-generation sequencing technologies” Nat Rev Genet 17,333-351.

### Methods

The following references include information useful for practicing the present method U.S. Pat. No. 10,266,894B2, “Methods and compositions for cDNA synthesis and single-cell transcriptome profiling using template switching reaction”; Picelli et al., 2013, “Smart-seq2 for sensitive full-length transcriptome profiling in single cells,” Nat Methods 10:1096-1098; Picelli et al., 2014, “Full-length RNA-Seq from single cells using Smart-seq2” Nat. Protocols 9:171-181: Picelli, “Single-cell RNA sequencing: The future of genome biology is now” (2017), RNA Biol; 14(5): 637-650); Saliba et al., “RNA-seq: advances and future challenges (2014). Nucleic Acids Res; 42:8845-60. Smart-seq2; and Plessy et al., 2010, “Linking promoters to functional transcripts in small samples with nanoCAGE and CAGEscan,” Nat Methods 7: 528-534 as relevant to Smart-seq-2. Each of the listed publications is incorporated herein by reference

### EXAMPLES

This section describes aspects of the Smart-seq-total method.

4-1. Cell Culture

HEK293T cells were cultured in complete DMEM high glucose medium (Gibco, ThermoFisher 11965092) supplemented with 5% Fetal Bovine Serum (ThermoFisher 16000044), 1 mM Sodium Pyruvate (ThermoFisher 11360070) and 100 μg/mL Penicillin/Streptomycin (ThermoFisher 15070063). They were collected 2h after passaging and sorted in either 96-well plates with 3uL lysis buffer or 384-well plates with 0.3 uL of lysis buffer in each well.

Human primary dermal fibroblasts were obtained from ATCC (ATCC® PCS-201-012™). Cells were cultured and passaged four times in Fibroblast Basal Medium (ATCC® PCS-201-030™) supplemented with 5 ng/mL rh FGF β, 7.5 mM L-glutamine, 50 ug/mL Ascorbic acid, 5ug/mL rh Insulin, and 1% Fetal Bovine Serum (Fibroblast Growth kit- low serum, ATCC® PCS-201-041™). MCF7 cells (ATCC® HTB22™) were cultured in complete DMEM high glucose medium (Gibco, ThermoFisher 11965092) supplemented with 10% Fetal Bovine Serum (ThermoFisher 16000044), 1 mM Sodium Pyruvate (ThermoFisher 11360070) and 100 μg/mL Penicillin/Streptomycin (ThermoFisher 15070063).

mESCs were maintained and differentiated as described previously^{35,36}. Briefly, mESCs were grown in serum-free 2i+LIF medium (complete medium: DMEM/F12 glutaMAX (Gibco, ThermoFisher 10565018) , 1% N2 supplement (Gemini Bio), 2% B27 supplement (Gemini Bio), 0.05% BSA fraction V (ThermoFisher, 15260037), 1% MEM-non-essential amino acids (c 11140050), and 110 μM 2-mercaptoethanol (Pierce); supplemented with MEK inhibitor PD0325901 (0.8 μM), GSK3β inhibitor CHIR99021 (3.3 μM) and 10 ng/mL mouse LIF (Gibco, PMC9484) in tissue culture (TC) dishes pretreated with 7.5 μg/ml polyL-ornithine (Sigma) and 5 μg/ml laminine (BD). To induce spontaneous embryoid body formation cells were washed with PBS, dissociated with StemPro Accutase (Gibco, ThermoFisher cat #A1110501), transferred to serum-rich medium (complete medium: DMEM/F12 glutaMAX (Gibco) , 1% N2 supplement (Gemini Bio), 2% B27 supplement (Gemini Bio), 0.05% BSA fraction V, 1% MEM-non-essential amino acids, and 110 μM 2-mercaptoethanol; supplemented with 10% FBS (ThermoFisher 10439001) and diluted to 10\hat{ }6 cells/mL. Each 10 uL of cell suspension were plated as a hanging drop in 10 cm² TC dishes (15-20 drops per dish). 10 uL of fresh serum-rich media was added to each drop on day 4 of the culture. Primed mESCs were collected 6 h after hanging drop culture. Embryoid bodies were collected and dissociated at days 4, 8 and 12 of culture.

### 4-2 Cell Sort

Lysis plates were prepared by dispensing 0.3 μL lysis buffer (4 U Recombinant RNase Inhibitor (RRI) (Takara Bio, 2313B), 0.12% Triton™ X-100 (Sigma, 93443-100ML), 1μM Smart-seq4 oligo-dT primer (5′-Biotin-CATAGTCTCGTGGGCTCGGAGATGTGTATAAGAGACAGT3OVN-3′ [SEQ ID NO:2]; IDT) into 384-well hard-shell PCR plates (Bio-Rad HSP3901) using Mantis liquid handler (Formulatrix). 96-well lysis plates were prepared with 3 μl lysis buffer. All plates were sealed with AlumaSeal CS Films (Sigma-Aldrich Z722634), spun down and snap-frozen on dry ice.

Cells were stained with calcein-AM and ethidium homodimer-1 (LIVE/DEAD® Viability/Cytotoxicity Kit, ThermoFisher L3224) and individual live cells were sorted in 384 well lysis plates using SONY sorter (5H8005) with 100um nozzle chip. Plates were spun down and stored at −80 degrees immediately after sorting.

4-3 Generation of Smart-seq-total Libraries

To facilitate cell lysis and denaturation of the RNA, plates were incubated at 72 degrees for 3 min, and immediately placed on ice afterwards. Next, 0.2 uL of polyA tailing mix, containing 1.25U E.coli PolyA (NEB M02765), 1.25× PolyA buffer (NEB), 1.25 mM ATPs (NEB) and 4U of RRI (Takara); were added to each samples. PolyA tailing was carried out for 15 minutes at 37° C. followed by 72° C. for 4 minutes. After polyA tailing plates were immediately placed on ice for 2-5 minutes. 1 uL of reverse transcription mix, containing 15U SuperScript II (ThermoFisher), 4U RRI (Takara), 1.5× First-Strand Buffer, 1.5 μM TSO (Exiqon, 5′-biotin-UCGUCGGCAGCGUCAGUUGUAUCAACUCAGACAUrGrG+G-3′ [SEQ ID NO:1]), 7.5 mM DTT, 1.5 M Betaine (Sigma, B0300-5VL), 10 mM MgCl₂ (Sigma, M1028-10X1ML) and 1.5 mM dNTPs (ThermoFisher, 18427013); was added to each well. Reverse transcription was carried out at 42° C. for 90 min, and terminated by heating at 85° C. for 5 min. Subsequently, 0.3 uL of TSO digestion buffer containing 1U Uracil-DNA glycosylase (UDG, NEB M02805) were added to each well. Plates were incubated for 30 minutes at 37° C. PCR preamplification was performed directly after TSO digestion by adding 3.2 μL of PCR mix, bringing reaction concentrations to 1× KAPA HiFi MIX (Roche), 0.5 μM Forward PCR primer (5′-TCGTCGGCAGCGTCAGTTGTATCAACT-3′ [SEQ ID NO:3]; IDT), 0.5 μM Reverse PCR primer (5′-GTCTCGTGGGCTCGGAGATGTG-3′ [SEQ ID NO:4]; IDT). PCR was cycled as follows: 1) 95° C. for 3 min, 3) 21 cycles of 98° C. for 20 s, 67° C. for 15 s and 72° C. for 6 min, and 4) 72° C. for 5 min. The amplified product was cleaned up using 0.8× ration of AMPure beads on Bravo liquid handler platform (Agilent). Concentrations of purified products were measured with a dye-fluorescence assay (Quant-iT PicoGreen dsDNA High Sensitivity kit; Thermo Fisher, Q33120) on a SpectraMax i3× microplate reader (Molecular Devices). Samples were then diluted to 0.2 ng/uL. To generate sequencing libraries, 1.5uL of diluted samples was amplified in a final volume 5 uL using 2× KAPA mix and 0.4 μl of 5 μM i5 indexing primer, 0.4 μl of 5 μM i7 indexing primer. PCR amplification was carried out using the following program: 1) 95° C. for 3 min, 2) 8 cycles of 98° C. for 20 s, 65° C. for 15 s and 72° C. for 1 min, and 4) 72° C. for 5 min.

4-4 Library Pooling, Ribosomal Sequence Digestion and Sequencing

After library preparation, wells of each library plate were pooled using a Mosquito liquid handler (UP Labtech). Pooling was followed by a purification using 1× AMPure beads (Fisher, A63881). Ribosomal sequences were digested using DASH as described in Gu et al., 2016³³. Briefly, 135 guides designed to target 5.8S and 45S rRNA sequence were combined with tracer RNA and assembled with Cas9 protein in 2:1 ratio. The assembled complexes were incubated with the sequencing library in 1× Cas9 buffer for 1h at 37° C. Following rRNA sequence digestion Cas9 was inactivated through incubation with proteinase K for 15 min at 50° C. Library was then purified twice, first using 1.2× and then 0.8× AMPure beads:DNA ratio. Library quality was assessed using capillary electrophoresis on a Fragment Analyzer (AATI), and libraries were quantified by qPCR (Kapa Biosystems, KK4923) on a CFX96 Touch Real-Time PCR Detection System (Biorad). Plate pools were normalized to 2 nM and equal volumes from 8 plates were mixed together to make the sequencing sample pool. A PhiX control library was spiked in at 10% before sequencing. Libraries were sequenced on the NovaSeq 6000 Sequencing System (Illumina) using 1×75 or 1×100-bp single-end reads (using custom Read 1 sequencing primer:5′-TCGGCAGCGTCAGTTGTATCAACTCAGACATGGG-3′ [SEQ ID NO:5]) and 2×12-bp index reads.

### 4-5 Data Processing

Sequences from the NovaSeq were de-multiplexed using bcl2fastq version 2.19.0.316. Reads were trimmed from polyA tails using cutadapt v 1.18 with the following parameters: -m 18-j 4-a AAAAAAAAAA-a TTTTTTTTTT. Reads were then aligned to the human (GRCh38) or mouse genome (GRCm38) using STAR_v2.7.0d³⁷ with the following parameters—outFilterMismatchNoverLmax 0.05—outFilterMatchNmin 18 --outFilterMatchNminOverLread 0--outFilterScoreMinOverLread 0—outMultimapperOrder Random. Reads mapping to multiple locations were assigned either to a location with the best mapping score or, in the case of equal multimapping score—to the genomic location randomly chosen as “primary”. Transcripts were counted using featureCounts v 1.6.1³⁸ with the following parameters-M—primary-s 1. GENCODE v32 and GENCODE M23³⁹—were used for human and mouse reads respectively. tRNA was quantified using high-confidence gene set obtained from GtRNA⁴⁰. To account for multimappers “primary” alignment reported by STAR was counted. For miRNA and tRNA all reads mapping either to arms or the stem loop were used to quantify the expression at the gene level.

4-5 Comparison of Smart-seq2 and Smart-seq-total

HEK293T cells were sorted in 96-well plates containing 3 uL of lysis buffer (as described above). The reaction volumes for Smart-seq4 were scaled 10 times compared to 384-plate format, i.e. RNA from each cell was polyadenylated in 5 uL, reverse transcribed in 15 uL and cDNA was pre-amplifying cDNA in 15 uL total volume. We retrieved Smart-seq2 data from (Picelli et al., 2013; GSE49321). Smart-seq2 and Smart-seq4-derived reads were mapped using STAR and counted using featureCounts as described above. Comparisons between protocols in FIG. 1b were generated on depth-normalized libraries, using 2.5 million randomly selected reads per library to compute expression levels (cpm).

### 4-6 Unsupervised Clustering and Dimensionality Reduction Analysis of Human Cell Types

Standard procedures for filtering, variable gene selection, dimensionality reduction and clustering were performed using the Seurat package version 3.1.4⁴¹. The parameters that were tuned on a per-cell type and feature type basis (resolution and number of principal components (PCs)) can be viewed in the Rmd files available on GitHub. Cells with fewer than 2000 detected genes and those with more than 2 Mio reads were excluded (a gene counts as detected if it has at least one read mapping to it). Counts were log-normalized for each cell using the natural logarithm of 1+counts per million. Variable genes were projected onto a low-dimensional subspace using principal component analysis. The number of principal components was selected on the basis of inspection of the plot of variance explained. A shared-nearest-neighbors graph was constructed on the basis of the Euclidean distance in the low-dimensional subspace spanned by the top principal components. Cells were visualized using a 2-dimensional t-distributed Stochastic Neighbor Embedding of the PC-projected data. Cells were assigned a cell cycle score using Seurat's CellCycleScoring( ) function and cell cycle markers described in⁴².

### 4-7 Clustering of Coding and Non-Coding Genes

Clusters of coding and non-coding genes shown in FIG. 3b were computed and visualized using DEGreport R package⁴³. Top 200 marker genes for each cell cycle phase and all non-coding genes with average expression In(cpm+1)>0.2 per in each phase were used. Gene expression values were normalized using variance stabilizing transformation⁴⁴. Further details can be viewed in the Rmd files available on GitHub.

4-8 Pre-Processing and Clustering of mESCs

Standard procedures for filtering, variable gene selection, dimensionality reduction and clustering were performed using the Seurat package version 3.1.4⁴¹. Cells with fewer than 1000 detected genes and those with more than 2 Mio reads were excluded (a gene counts as detected if it has at least one read mapping to it). Counts were log-normalized for each cell using the natural logarithm of 1+counts per million. Variable genes were projected onto a low-dimensional subspace using principal component analysis. The number of principal components was selected on the basis of inspection of the variance explained plot. A shared-nearest-neighbors graph was constructed on the basis of the Euclidean distance in the low-dimensional subspace spanned by the top principal components. Cells were visualized using Uniform manifold Approximation and Projection (UMAP) algorithm⁴⁵ of the PC-projected data. Clusters were annotated based on the expression of known marker genes corresponding to one of the three germ layers. Cells were assigned a cell cycle score using Seurat's CellCycleScoring( )function and cell cycle markers described in⁴².

### 4-9 Developmental Trajectory Inference of EB Differentiation

Developmental trajectory of mESC differentiation was inferred using PAGA through dynoverse wrapper⁴⁶. Pseudotime coordinates computed from the trajectory were appended to Seurat object and further used to generate FIGS. 2f-i.

### 4-10 Code Availability

AH code used for analysis is available on GitHub (https://github.com/aisakova/).

### Terms

As used herein, “ncRNA” is non-protein-coding RNA.

As used herein, “RNA” may refer to an individual RNA molecule or to a population or mixture of RNA molecules; the sense in which “RNA” is used will be apparent from context to one of ordinary skill in the art.

As used herein, “poly(A)-plus RNA” refers to RNA with a poly(A) tail consisting of multiple adenosine monophosphates. Poly(A)-plus RNA include mRNA.

As used herein, “poly(A)-minus RNA” refers to RNA that does not comprise a poly(A) tail and includes miRNA, IncRNA, snoRNA, Y RNA, snRNA, SRP RNA, RNA7SK, RN7SL1 and piRNA, tRNA and rRNA. Some transcripts of histone encoding genes are poly(A)-minus.

“+G” designates a locked nucleic acid (LNA)-modified guanosine. Locked nucleic acids are well known in the art. A locked nucleic acid is a modified RNA monomer having a methylene bridge bond linking the 2′ oxygen to the 4′ carbon of the RNA pentose ring, fixing the pentose ring in the 3′-endo conformation.

V denotes A or C or G (see WIPO Standard ST.25 (1998), Appendix 2).

### REFERENCES

A. Alina lsakova, Norma Neff, Stephen R. Quake, “Single cell profiling of total RNA using Smart-seq-total” bioRxiv 2020.06.02.131060; doi: https:// doi.org/10.1101/2020.06. 02.131060 (published after priority date) is incorporated herein by reference.
1. Mereu et al., Benchmarking single-cell RNA-sequencing protocols for cell atlas projects. Nat. Biotechnol. (2020). doi:10.1038/s41587-020-0469-4
2. The Tabula Muris Consortium, Overall coordination, Logistical coordination, Organ collection and processing, Library preparation and sequencing, Computational data analysis, Cell type annotation, Writing group, Supplemental text writing group & Principal investigators. Single-cell transcriptomics of 20 mouse organs creates a Tabula Muris. Nature 562, 367-372 (2018).
3. Regev et al., Human Cell Atlas Meeting Participants. The Human Cell Atlas. eLife 6, e27041 (2017).
4. Kester, L. & van Oudenaarden, A. Single-Cell Transcriptomics Meets Lineage Tracing. Cell Stem Cell 23, 166-179 (2018).
5. Melton, C., Judson, R. L. & Blelloch, R. Opposing microRNA families regulate self-renewal in mouse embryonic stem cells. Nature 463, 621-626 (2010).
6. Flynn, R. A. & Chang, H. Y. Long Noncoding RNAs in Cell-Fate Programming and Reprogramming. Cell Stem Cell 14, 752-761 (2014).
7. Isakova, A., Fehlmann, T., Keller, A. & Quake, S. R. A mouse tissue atlas of small non-coding RNA. (biorxiv, 2018). doi:10.1101/430561
8. Gil, N. & Ulitsky, I. Regulation of gene expression by cis-acting long non-coding RNAs. Nat. Rev. Genet. 21, 102-117 (2020).
9. Pennisi, E. ENCODE Project Writes Eulogy for Junk DNA. Science 337, 1159-1161 (2012).
10. Wang, N., Zheng, J., Chen, Z., Liu, Y., Dura, B., Kwak, M., Xavier-Ferrucio, J., Lu, Y.-C., Zhang, M., Roden, C., Cheng, J., Krause, D. S., Ding, Y., Fan, R. & Lu, J. Single-cell microRNA-mRNA co-sequencing reveals non-genetic heterogeneity and mechanisms of microRNA regulation. Nat. Commun. 10, 95 (2019).
11. Faridani, O. R., Abdullayev, I., Hagemann-Jensen, M., Schell, J. P., Lanner, F. & Sandberg, R. Single-cell sequencing of the small-RNA transcriptome. Nat. Biotechnol. 34, 1264-1266 (2016).
12. Wang, Y., Xu, Z., Jiang, J., Xu, C., Kang, J., Xiao, L., Wu, M., Xiong, J., Guo, X. & Liu, H. Endogenous miRNA Sponge lincRNA-RoR Regulates Oct4, Nanog, and Sox2 in Human Embryonic Stem Cell Self-Renewal. Dev. Cell 25, 69-80 (2013).
13. Fan, X., Zhang, X., Wu, X., Guo, H., Hu, Y., Tang, F. & Huang, Y. Single-cell RNA-seq transcriptome analysis of linear and circular RNAs in mouse preimplantation embryos. Genome Biol. 16, 148 (2015).
14. Liu, S. J., Nowakowski, T. J., Pollen, A. A., Lui, J. H., Horlbeck, M. A., Attenello, F. J., He, D., Weissman, J. S., Kriegstein, A. R., Diaz, A. A. & Lim, D. A. Single-cell analysis of long non-coding RNAs in the developing human neocortex. Genome Biol. 17, 67 (2016).
15. Verboom, K., Everaert, C., Bolduc, N., Livak, K. J., Yigit, N., Rombaut, D., Anckaert, J., Lee, S., Veno, M. T., Kjems, J., Speleman, F., Mestdagh, P. & Vandesompele, J. SMARTer single cell total RNA sequencing. Nucleic Acids Res. 47, e93—e93 (2019).
16. Picelli, S., Bjorklund, A. K., Faridani, O. R., Sagasser, S., Winberg, G. & Sandberg, R. Smart-seq2 for sensitive full-length transcriptome profiling in single cells. Nat. Methods 10, 1096-1098 (2013).
17. Zhu, Y. Y., Machleder, E. M., Chenchik, A., Li, R. & Siebert, P. D. Reverse transcriptase template switching: a SMART approach for full-length cDNA library construction. BioTechniques 30, 892-897 (2001).
18. Turchinovich, A., Surowy, H., Serva, A., Zapatka, M., Lichter, P. & Burwinkel, B. Capture and Amplification by Tailing and Switching (CATS): An ultrasensitive ligation-independent method for generation of DNA libraries for deep sequencing from picogram amounts of DNA and RNA. RNA Biol. 11, 817-828 (2014).
19. Lee, Y.-H., Hsueh, Y.-W., Peng, Y.-H., Chang, K.-C., Tsai, K.-J., Sun, H. S., Su, I.-J. & Chiang, P.-M. Low-cell-number, single-tube amplification (STA) of total RNA revealed transcriptome changes from pluripotency to endothelium. BMC Biol. 15, (2017).
20. Bueno, M. J. & Malumbres, M. MicroRNAs and the cell cycle. Biochim. Biophys. Acta BBA—Mol. Basis Dis. 1812, 592-601 (2011).
21. Otto, T., Candido, S. V., Pilarz, M. S., Sicinska, E., Bronson, R. T., Bowden, M., Lachowicz, I. A., Mulry, K., Fassl, A., Han, R. C., Jecrois, E. S. & Sicinski, P. Cell cycle-targeting microRNAs promote differentiation by enforcing cell-cycle exit. Proc. Natl. Acad. Sci. 114, 10660-10665 (2017).
22. Harris, M. E., Bohni, R., Schneiderman, M. H., Ramamurthy, L., Schümperli, D. & Marzluff, W. F. Regulation of histone mRNA in the unperturbed cell cycle: evidence suggesting control at two posttranscriptional steps. Mol. Cell. Biol. 11, 2416-2424 (1991).
23. Wu, R. S. & Bonner, W. M. Separation of basal histone synthesis from S-phase histone synthesis in dividing cells. Cell 27, 321-330 (1981).
24. Höpfl G., Gassmann M. & Desbaillets I. Differentiating Embryonic Stem Cells into Embryoid Bodies. 254, (Humana Press, 2004).
25. Guttman et al. lincRNAs act in the circuitry controlling pluripotency and differentiation. Nature 477, 295-300 (2011).
26. Lee, S., Seo, H.-H., Lee, C. Y., Lee, J., Shin, S., Kim, S. W., Lim, S. & Hwang, K.-C. Human Long Noncoding RNA Regulation of Stem Cell Potency and Differentiation. Stem Cells Int. 2017, 1-10 (2017).
27. Gulati et al., Single-cell transcriptional diversity is a hallmark of developmental potential. Science 367, 405-411 (2020).
28. Pekkanen-Mattila, M., Pelto-Huikko, M., Kujala, V., Suuronen, R., Skottman, H., Aalto-Setälä, K. & Kerkela, E. Spatial and temporal expression pattern of germ layer markers during human embryonic stem cell differentiation in embryoid bodies. Histochem. Cell Biol. 133, 595-606 (2010).
29. van den Brink, S. C., Alemany, A., van Batenburg, V., Moris, N., Blotenburg, M., Vivié, J., Baillie-Johnson, P., Nichols, J., Sonnen, K. F., Martinez Arias, A. & van Oudenaarden, A. Single-cell and spatial transcriptomics reveal somitogenesis in gastruloids. Nature (2020). doi:10.1038/s41586-020-2024-3.
30. Han, X., Chen, H., Huang, D., Chen, H., Fei, L., Cheng, C., Huang, H., Yuan, G.-C. & Guo, G. Mapping human pluripotent stem cell differentiation pathways using high throughput single-cell RNA-sequencing. Genome Biol. 19, 47 (2018).
31. Wolf et al., PAGA: graph abstraction reconciles clustering with trajectory inference through a topology preserving map of single cells. Genome Biol. 20, 59 (2019).
32. Wulf, M. G., Maguire, S., Humbert, P., Dai, N., Bei, Y., Nichols, N. M., Corrêa, I. R. & Guan, S. Non-templated addition and template switching by Moloney murine leukemia virus (MMLV)-based reverse transcriptases co-occur and compete with each other. J. Biol. Chem. 294, 18220-18231 (2019).
33. Gu, W., Crawford, E. D., O'Donovan, B. D., Wilson, M. R., Chow, E. D., Retallack, H. & DeRisi, J. L. Depletion of Abundant Sequences by Hybridization (DASH): using Cas9 to remove unwanted high-abundance species in sequencing libraries and molecular counting applications. Genome Biol. 17, 41 (2016).
34. Marzluff, W. F., Wagner, E. J. & Duronio, R. J. Metabolism and regulation of canonical histone mRNAs: life without a poly(A) tail. Nat. Rev. Genet. 9, 843-854 (2008).
35. Höpfl, G., Gassmann, M. & Desbaillets, I. in Germ Cell Protoc. 254, 079-098 (Humana Press, 2004).
36. Rada-Iglesias, A., Bajpai, R., Swigut, T., Brugmann, S. A., Flynn, R. A. & Wysocka, J. A unique chromatin signature uncovers early developmental enhancers in humans. Nature 470, 279-283 (2011).
37. Dobin, A., Davis, C. A., Schlesinger, F., Drenkow, J., Zaleski, C., Jha, S., Batut, P., Chaisson, M. & Gingeras, T. R. STAR: ultrafast universal RNA-seq aligner. Bioinformatics 29, 15-21 (2013).
38. Liao, Y., Smyth, G. K. & Shi, W. featureCounts: an efficient general purpose program for assigning sequence reads to genomic features. Bioinformatics 30, 923-930 (2014).
39. Frankish et al., GENCODE reference annotation for the human and mouse genomes. Nucleic Acids Res. 47, D766—D773 (2019).
40. Kozomara, A. & Griffiths-Jones, S. miRBase: annotating high confidence microRNAs using deep sequencing data. Nucleic Acids Res. 42, D68—D73 (2014).
41. Stuart, T., Butler, A., Hoffman, P., Hafemeister, C., Papalexi, E., Mauck, W. M., Hao, Y., Stoeckius, M., Smibert, P. & Satija, R. Comprehensive Integration of Single-Cell Data. Cell 177, 1888-1902.e21 (2019).
42. Tirosh et al., Dissecting the multicellular ecosystem of metastatic melanoma by single-cell RNA-seq. Science 352, 189-196 (2016).
43. Pantano Lorena. DEGreport: Report of DEG analysis. (2019). at <http://lpantano.github.io/DEGreport/<
44. Love, M. I., Huber, W. & Anders, S. Moderated estimation of fold change and dispersion for RNA-seq data with DESeq2. Genome Biol. 15, 550 (2014).
45. McInnes, L., Healy, J. & Melville, J. UMAP: Uniform Manifold Approximation and Projection for Dimension Reduction. ArXiv180203426 Cs Stat (2018). at <http://arxiv.org/abs/1802.03426>
46. Saelens, W., Cannoodt, R., Todorov, H. & Saeys, Y. A comparison of single-cell trajectory inference methods. Nat. Biotechnol. 37, 547-554 (2019).
47. Cloonan et al., 2008, “Stem cell transcriptome profiling via massive-scale mRNA sequencing. Nat Methods 5: 613-619. doi:10.1038/nmeth.1223. PubMed: 18516046.
48. Plessy et al., 2010, Linking promoters to functional transcripts in small samples with nanoCAGE and CAGEscan. Nat Methods 7: 528-534. doi:10.1038/nmeth.1470.

All publications and patent documents cited herein are incorporated herein by reference as if each such publication or document was specifically and individually indicated to be incorporated herein by reference. Citation of publications and patent documents (patents, published patent applications, and unpublished patent applications) is not intended as an admission that any such document is pertinent prior art, nor does it constitute any admission as to the contents or date of the same.

1. A method for preparing DNA complementary to poly(A)-minus RNA comprising the steps of:
i) treating the poly(A)-minus RNA with
(a) poly(A) polymerase (PAP) to add a 3′ poly(A) tail to the poly(A)-minus RNA, thereby producing poly(A)-plus RNA, or
(b) polynucleotide transferase to add a homopolynucleotide poly(N), where poly(N) is selected from poly(A), poly(C), poly(G) and poly(U), thereby producing poly(N)-plus RNA;
ii) annealing a cDNA synthesis primer comprising oligo(dT) to the poly(A)-plus RNA produced in (i)(a), or annealing a cDNA synthesis primer comprising oligo(dN′) to the poly(N)-plus RNA produced in (i)(b), wherein N′ [“N prime”] is a nucleotide that basepairs with N, and synthesizing a first cDNA strand, thereby producing an RNA-cDNA intermediate;
iii) conducting a template switching reaction by contacting the RNA-cDNA intermediate with a template switching oligonucleotide (TSO) under conditions suitable for extension of the first cDNA strand, rendering the first cDNA strand additionally complementary to the TSO, wherein the TSO comprises at least two (2) deoxyuridine nucleotides
iv) degrading the TSO.

2. The method of claim 1, further comprising (v) amplifying sequence from the first cDNA strand to produce a pool of amplicons.

3. The method of claim 2, further comprising (vi) depleting high-abundance RNA species from the pool of amplicons.

4. The method of claim 3, wherein the high-abundance RNA species comprises ribosomal RNA.

5. The method of claim 1, wherein in step (iv) the TSO is degraded by treatment with uracil DNA glycosylase (UDG), and optionally with a combination of UDG and endonuclease VIII.

6. The method of claim 1 wherein in step (i)(a) a mixture comprising poly(A)-minus RNA and polyadenylated RNA is treated with poly(A) polymerase (PAP) to add a 3′ poly(A) tail to the poly(A)-minus RNA and to the polyadenylated RNA, thereby producing the poly(A)-plus RNA.

7. The method of claim 6, wherein the mixture comprises RNA from one single cell, optionally a human cell.

8. The method of claim 7, wherein the mixture comprises total RNA from a single cell.

9. The method of claim 6, wherein the mixture comprises RNA from human cells.

10. The method of claim 2, wherein the step of amplifying comprises associating the sequence from the first cDNA strand with one or more sequence elements selected from adaptors, indexing sequences, oligonucleotide binding sequences and barcodes.

11. The method of claim 2, further comprising sequencing amplicons produced in step (v).

12. The method of claim 2, wherein steps (i)-(iv) or steps (i)-(v) are carried out in the same compartment.

13. (canceled)

14. A template switching oligonucleotide (TSO) comprising at least two (2) deoxyuridine nucleotides, at least two ribonucleotide residues.

15. The TSO of claim 14 that is 30 to 50 nucleotides in length and comprises three (3) to ten (10) deoxyuridine nucleotides.

16. The TSO of claim 15 that comprises rGrG+G at its 3′ end, where +G is a locked nucleic acid.

17. The TSO of claim 14, wherein
i) the TSO has at least 2, at least 3, at least 4, at least 5, at least 6, at least 7, at least 8, at least 9 , or at least 10 deoxyuridine nucleotides;
ii) at least 10%, at least 15% or at least 20% of the nucleotides in the TSO are deoxyuridine; or
iii) deoxyuridine nucleotides are spaced or positioned in the TSO such that following fragmentation of the TSO by deamination of all uridine nucleotides no remaining fragment of the TSO is longer than 10 nucleotides, no remaining fragment of the TSO is longer than 9 nucleotides, or no remaining fragment of the TSO is longer than 8 nucleotides.

18. A method of identifying or distinguishing human cell types based on the abundance of non-coding transcripts comprising (i) determining an expression profile of at least one, preferably at least two, poly(A)-minus RNA type selected from miscRNA, lncRNA, snoRNA, miRNA, snRNA and tRNA in a cell(s) and (ii) matching the profile to a reference profile characteristic of a human cell type.

19. The method of claim 12, wherein cell lysis prior to step (i) is carried out in the same compartment.

20. The TSO of claim 16, wherein the TSO is biotinylated at the 5′ terminus.


SEQUENCE LISTING (8 sequences)

SEQ ID NO: 1 - RNA, 37 nt, Artificial Sequence
  Description of Artificial Sequence Synthetic oligonucleotide
ucgucggcagcgucaguuguaucaacucagacauggg

SEQ ID NO: 2 - DNA, 70 bp, Artificial Sequence
  Description of Artificial Sequence Synthetic primer
  modified_base (70)..(70): a, c, t, g, unknown or other
catagtctcgtgggctcggagatgtgtataagagacagttttttttttttttttttttttttttttttvn

SEQ ID NO: 3 - DNA, 27 bp, Artificial Sequence
  Description of Artificial Sequence Synthetic primer
tcgtcggcagcgtcagttgtatcaact

SEQ ID NO: 4 - DNA, 22 bp, Artificial Sequence
  Description of Artificial Sequence Synthetic primer
gtctcgtgggctcggagatgtg

SEQ ID NO: 5 - DNA, 34 bp, Artificial Sequence
  Description of Artificial Sequence Synthetic primer
tcggcagcgtcagttgtatcaactcagacatggg

SEQ ID NO: 6 - RNA, 10 nt, Artificial Sequence
  Description of Artificial Sequence Synthetic oligonucleotide
aaaaaaaaaa

SEQ ID NO: 7 - DNA, 10 bp, Artificial Sequence
  Description of Artificial Sequence Synthetic oligonucleotide
tttttttttt

SEQ ID NO: 8 - RNA, 12 nt, Artificial Sequence
  Description of Artificial Sequence Synthetic oligonucleotide
aaaaaaaaaaaa